(19) United States
(12) Patent Application Publication
(10) Pub. No.: US 2006/0188941 A1
(43) Pub. Date: Aug. 24, 2006

(54) High throughput screening assays utilizing affinity binding of green fluorescent protein

(76) Inventors: William W. Ward, Metuchen, NJ (US); Catherine Thomson, Edinburgh (GB)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/360,863

(22) Filed: Feb. 23, 2006

Related U.S. Application Data
(62) Division of application No. 10/161,169, filed on May 31, 2002.
(60) Provisional application No. 60/295,184, filed on Jun. 1, 2001.

(51) Int. Cl. G01N 33/53; G06F 19/00

(52) U.S. Cl. 435/7.1; 702/19

(57) ABSTRACT

Novel methods of detecting fluorescent proteins are described. The methods result in vastly improved signal-to-noise ratios in assays measuring fluorescence of a fluorescent protein specifically by employing a unique trapping step to microconcentrate the fluorescent protein and by using improved optical techniques. The trapping step may be a chemical or physical process or a combination thereof leading to substantial microconcentration of the fluorescent protein with concomitant removal of contaminants or interfering compounds. The methods are readily adaptable to high throughput screening and can be engineered for use with a wide variety of assays currently using microplate readers. Green fluorescent protein and fluorescent coral proteins are among preferred fluorescent proteins for the methods.

### CROSS REFERENCE TO RELATED APPLICATION

This application claims benefit of U.S. Provisional Application No. 60/295,184, filed Jun. 1, 2001, the entirety of which is incorporated by reference herein.

### FIELD OF THE INVENTION

This invention relates to the field of pharmaceutical and biotechnology research and development. Specifically, this invention provides methods and devices for rapid screening of compounds with potential as therapeutic agents or research tools.

### BACKGROUND OF THE INVENTION

Various scientific and scholarly articles are referred to in parentheses throughout the specification. These articles are incorporated by reference to describe the state of the art to which this invention pertains. In addition, any sequences referred to by Accession Number of a publicly accessible database are incorporated by reference herein.

The screening of potential candidates for therapeutic agents is critical to maintaining a full pipeline of products for the pharmaceutical industry. Despite its importance to the industry and, indirectly, to the public, drug screening is often a bottleneck in the development of new drugs to alleviate conditions ranging from the common cold to cancer. Traditional methods have been either labor intensive, time-consuming, or too expensive. In addition, many potentially valuable therapeutic agents may be missed because of inadequate screening assays.

Rapid technological changes in several fields have led to an increasing demand for high throughput screening (HTS) assays. These changes include, for example, the following:

1. Genomics: As more DNA sequences are determined, more potential therapeutic targets develop as gene functions are learned and associations with disease are made. For the first time in history, the entire genetic code of many organisms will be available to researchers. The predominance of gene sequences that are being reported has generated many new targets for reporter gene assays. Such assays are used to measure gene expression, or aspects thereof, e.g. presence or absence of a gene of interest, relative promoter activity, proper processing and subcellular localization. Examples of currently used reporter assays include antibiotic resistance genes, enzyme activities and color- or light-producing proteins—all of which are quickly and easily measured. Chloramphenicol acetyl transferase (‘CAT’), β-galactosidase, β-glucuronidase (‘GUS’) and alkaline phosphatase, and luciferase have all been widely used in this capacity.

2. Computers: Faster, smaller and more networked, the widespread use of computers and massive databases allows even greater capacity to compare properties of known and unknown chemicals as potential therapeutic agents, and also to search for new potential targets for drug therapy. Automated data collection and analysis greatly speed up the work of screening.

3. Combinatorial chemistry: The ability to rapidly and easily synthesize a multitude of compounds with different properties for screening is especially useful for generating families of compounds related to lead compounds or ‘hits’ from early rounds of screening.

4. Mechanical Technological Advances: Robotics, miniaturization and microfluidics have all advanced the art significantly in recent years. The reduction of the need for human labor, the use of smaller quantities and the reduced need for space along with the ability to use exponentially smaller volumes have all dramatically enabled the development of HTS assay systems.

5. Economic factors: Market forces are powerful influences on technology development and uses. Pressure to reduce the cost of drugs also forces the cost of drug development down. To survive, pharmaceutical companies must look for more efficient, lower cost methods of drug discovery. HTS is one such method being widely utilized.

The advances in each of these areas have helped to cooperatively drive forward the state of the art in HTS in a concerted fashion. However all these advances have created a situation where the potential ability to rapidly and cost effectively screen chemical compounds for ‘activity’ on a multitude of theoretical targets has outstripped the basic biological strategies and principles of assay development. What is the ‘activity’ to be measured?

Currently many HTS assays are in use. For example, many use high throughput-type assays to measure specific affinity-ligand interactions. In a typical application, multi-well plates are used. Cells of interest are contained within the wells of the plate. The cells are incubated in the presence of test compounds, designed to bind specifically to a particular target in the cell. After the interaction between the target cell and the test compound, the unbound material is optionally removed by a washing or separation step, then a measurement is made of the amount of test compound which specifically bound to its receptor. Measurement is by use of radiolabeled compounds, or by use of fluorescent labels. Only those compounds which are directly involved with the receptor-ligand interaction can be screened by such an approach. Other high throughput screening assays measure enzyme activity inhibitors, while still others measure the agonist or antagonist activity of receptor-mediated intracellular processes.

Such methods do allow for rapid screening, however they often suffer from problems such as high background levels, or low levels of signal—this a particularly problematic situation, especially when the signal-to-noise ratio is low because it can result it both false positives and false negatives. In addition to the problems of high background and low signal which plague many assay systems, assays which are based on radiolabeled compounds pose additional hazards to those who work with them and are a waste disposal problem.

Considering the expense associated with drug screening and the cost of moving a screened compound forward to the next phase of drug development, the cost of falsely identifying a potential compound as useful is significant. Of potentially even greater cost to both the pharmaceutical company and to the public is the cost of a false negative. One useful and novel compound missed due to high background or low signal has huge, though unmeasured effect. In addition to the billions of potential dollars in lost revenue, and lives not saved, it may cause the researchers to miss an entire class of compounds which may have been useful to treat other conditions.

The use of Green Fluorescent Protein (GFP) as a reporter of gene expression was first brought to fruition by Chalfie et al. (1994). They demonstrated that the GFP from Aequoria victoria was more useful to monitor gene activity and protein distribution than previously-used systems such as those encoding fusions with luciferase or β-galactosidase, since the latter systems required exogenously added cofactors or substrates. In living systems, GFP was expressed, and upon irradiating cells with blue or near-UV light, would fluoresce brightly to reveal cellular or subcellular localization of expression. The GFP was shown to be nontoxic to the cells—even when constitutively expressed via a strong promoter.

Since that time, several GFPs from different organisms have been identified, and in some cases isolated or cloned (Ward chapter, Patent App no., Bryan patent). Other related fluorescent proteins have also been identified (Matz et al.). All of these GFPs have potential use as reporters of gene expression.

Most of the applications have been restricted to microscopic examination of transformed cells. In these applications, GFP is an excellent spatial reporter for gene induction or expression, protein trafficking, and real time cellular events. In such applications, the human eye or a sophisticated computer program does the work of distinguishing desired signal from noise.

One problem which has plagued assays using the green fluorescent proteins to date is that of ‘noise’, most particularly in non-microscopic assays. Despite the signal created by the emission of the GFP, there are numerous sources of background fluorescence, autofluorescence, scatter and other interference in the assays in which it has been used. These sources include for example cellular components and debris, and the glass- and or plastic-ware used.

Another problem common in such assays is low signal strength. In many cases the intensity of the light used for excitation is limited by the anticipated noise. The spectral properties of the GFPs used to date are also somewhat limiting, in that large amounts of expression are often needed to overcome detection limits.

In summary, the GFP reporter assays that have been attempted for high throughput screening have tended to suffer from low signal-to-noise ratios. Since currently available HTS assays tend to suffer from limitations due to high background, scatter, and other noise, and/or from low signal strength, the development of an HTS assay system wherein the signal-to-noise ratio is several orders of magnitude greater than existing assays would be quite useful and significant.

### SUMMARY OF THE INVENTION

The present invention provides methods for increasing the signal-to-noise ratio in assays involving fluorescent proteins. The invention further provides assays to identify potentially therapeutic compounds via high throughput screening in cells or cell-free systems.

In one embodiment, a method for increasing the signal-to-noise ratio in assays measuring the properties of fluorescence proteins is provided. The method comprises the following steps: providing a fluorescent protein (FP) in an assay reaction; trapping the FP by use of a trapping step for separating the FP from one or more interfering components; concentrating the trapped FP into a compact area; irradiating the trapped, concentrated FP with a light source at an excitation wavelength; and detecting an emitted light intensity at an emission wavelength.

In one embodiment, the FP is a green fluorescent protein (GFP). The GFP can be from any source, with Aequoria victoria and Renilla spp. being particularly useful. In a preferred embodiment the GFP is from A. victoria or from R. reniformis. In some embodiments the GFP is produced from a genetically manipulated gene.

In other embodiments, the FP is a fluorescent protein from another organism. For instance, the red coral fluorescent protein DsRed is particularly suitable for use in the invention.

In one embodiment, the trapping step is a chemical means for binding the FP, to separate it from other components, particularly interfering components in the reaction mixture. In one embodiment the trapping step results in a concomitant concentration of the FP by a factor of from about 1-fold to about 1000-fold. In a preferred embodiment, the trapping step results in a concentration factor from about 10³-fold up to about 10⁶-fold or greater. In other embodiments, a concentration step, such as are known in the art, is used in conjunction with the trapping step.

The trapping step in various embodiments comprises molecular interactions, for example: metal ion affinity binding, ion exchange interactions, or antigen-antibody interactions. In some embodiments, the interacting portions or domains of the GFP involved in the molecular interactions may be exogenous to a native GFP protein, e.g. they may be the result of genetic modification of a gene encoding a GFP molecule.

Reducing the interfering compounds and concentrating the sample allow for a decrease in noise and allow the use of higher intensity excitation wavelength irradiation and generation of higher signals. This results in greatly increased signal-to-noise ratios. In a preferred embodiment, the signal-to-noise ratio is increased about one to many orders of magnitude.

In another embodiment of the present invention, a method for quantifying a fluorescent protein (FP) produced in a cell-based or cell-free expression assay system is provided. The steps of the method are as follows: providing a reaction medium in which to quantify a FP produced during an assay; trapping the produced FP by use of a trapping step for separating the produced FP from one or more interfering components; concentrating the trapped FP into a compact area; irradiating the trapped, concentrated FP with a light source at an excitation wavelength; detecting an emitted light intensity at an emission wavelength; and quantifying the produced FP as a function of the emitted light intensity of the trapped FP.

In a preferred embodiment, the FP is a GFP. The method can be used for quantifying the GFP when it is produced in reporter gene—type assays. In one embodiment, screening is based on the promoter-driven expression of GFP. The GFP expressed in these assays exhibits an increased and more uniform level of fluorescence after a step for trapping and concentration, than that exhibited in previously known HTS assays. A preferred GFP is based on that from A. victoria, more preferred is a GFP based on that from a Renilla spp.

Where the GFP is produced in cells, the method may involve an optional lysis step. From such a step a functional GFP is recovered, while cellular membranes and other cellular components may be disrupted or disintegrated.

In one embodiment, the method uses a high intensity or ultra-high intensity light source to irradiate the trapped and concentrated GFP. Such light sources are known in the art, for example argon lasers. The removal of interfering compounds allows for the light intensity to be greatly increased without risk of elevated noise due to scattering or autofluorescence. Under such conditions, where the GFP is trapped and concentrated, with the exclusion of scattering and autofluorescent contaminants, the true GFP signal is proportional to the intensity of the light at the excitation wavelength.

In a preferred embodiment, the method is automated for quantifying a plurality of assays. The automation of the method is by methods such as are known in the art for automatically processing the assays—handling and transporting samples and reactants, performing incubations, mixing, removal or addition of components, quantifying reactants, recording data appropriately. In a preferred embodiment, the automation is developed as part of a high throughput screening system. In one embodiment, the assay is miniaturized to allow smaller volumes and faster manipulation of samples, with lower consumption of reactants. Miniaturization also facilitates significantly increasing the light intensity for the excitation wavelength.

In is another object of the present invention to provide a method for quantifying the activity of a nucleic acid expression system in a cell-based or cell-free system. The method comprises the following steps: incubating an assay mixture containing an expression system comprising a nucleic acid sequence encoding a functional GFP operably-linked to an expression regulatory element, under suitable conditions for expression of the GFP; trapping the expressed GFP by use of a trapping step for separating the produced GFP from one or more interfering components; concentrating the trapped GFP into a compact area; irradiating the trapped, concentrated GFP with a light source at an excitation wavelength; detecting an emitted light intensity at an emission wavelength; and, quantifying the activity of the expression system as a function of the emitted light intensity of the trapped FP.

In one embodiment, the nucleic acid expression system is an in vivo expression system, in other embodiments it is a in vitro transcription, in vitro translation or in vitro transcription/translation system. In one embodiment a DNA sequence is being expressed to produce an RNA molecule. In another embodiment, a protein is being produced either directly from an mRNA, or indirectly from a DNA sequence, including a cDNA sequence or an artificial sequence.

In one embodiment of this method, the increased sensitivity of the assay allows for the detection of more subtle differences among the various biological regulatory machinery and structural components which are involved in the nucleic acid expression system.

In another aspect of the instant invention a similar method can be used to screen mutants which possess alterations in the activity of a nucleic acid expression system. The method is particularly useful for finding more subtle mutants which cannot be detected by traditional ‘on/off’ screens and the like. Such subtle mutants may be useful for understanding the kinds of nonlethal mutations important to agricultural improvement programs, or alternatively specific genetic disease processes.

In another aspect, the present invention features an instrument for the measurement of fluorescence produced by the GFP present in these assays. This instrument provides excitation energy at a much greater intensity than previously known and used in non-microscopic fluorescence-measuring instruments.

In yet another aspect, the present invention features GFP standards for use in HTS assay systems and other systems which measure GFP fluorescence. Methods for preparing and using such standards are provided.

Yet another aspect of the invention features combinations of the aforementioned elements into a HTS assay system with greatly enhanced signal-to-noise ratios, greater sensitivity, and improved quantitation relative to existing assays. Other and further features and advantages of the invention will become apparent from the following description.

### BRIEF DESCRIPTION OF THE DRAWINGS

FIG. 1: Color photomicrograph (200×) showing 5 μm C₄-derivatized silica beads with wild-type GFP bound by hydrophobic interaction.

FIG. 2: Color photomicrograph (100×) depicting DEAE Sepaharose Fast Flow chromatography beads (average size=90 μm) with high, medium and low relative amounts of wild-type GFP (appearing as white, green or teal fluorescence respectively) bound by ionic interaction with the DEAE functional group.

FIG. 3: Color photomicrograph (400×) (of whole, live E. coli BL-21 cells expressing red coral fluorescent protein, DsRed 1 (clontech). Individual bacteria in the field have average diameter of about 1 μm.

FIG. 4: Color photomicrograph (200×) showing 5 μm C₄-derivatized silica beads with DsRed 1 fluorescent protein bound through hydrophobic interaction. The DsRed 1 protein was produced in E. coli BL-21 cells.

FIG. 5: Color photomicrograph (200×) showing a mixture of 5 μm C₄-derivatized silica beads with either DsRed 1 fluorescent protein or wild-type GFP, each bound through hydrophobic interaction.

FIG. 6: Color photomicrograph (400×) showing a similar mixture of silica beads at greater magnification to reveal detail.

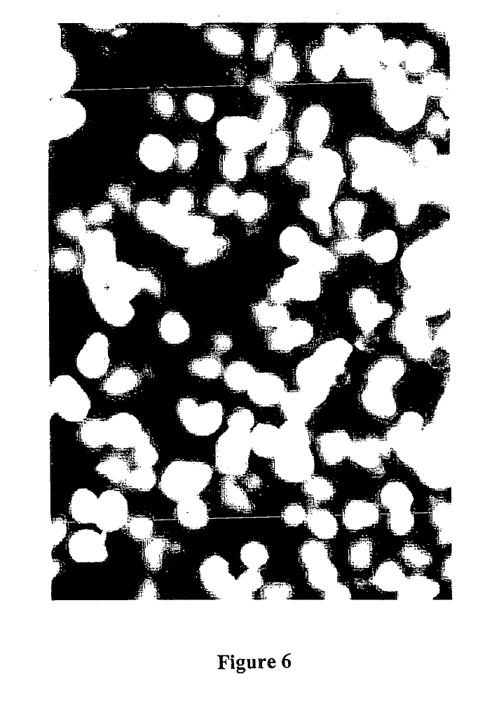

### DETAILED DESCRIPTION OF THE INVENTION

The present invention provides, in one aspect, methods for increasing the signal-to-noise ratio in assays involving fluorescent proteins. A method for increasing the signal-to-noise ratio in assays measuring the properties of fluorescence proteins is provided. The method comprises the following steps: providing a fluorescent protein (FP) in an assay reaction; trapping the FP by use of a trapping step for separating the FP from one or more interfering components; concentrating the trapped FP into a compact area; irradiating the trapped, concentrated FP with a light source of high intensity at an excitation wavelength; and detecting an emitted light intensity at an emission wavelength.

In one embodiment, the FP is a green fluorescent protein (GFP). The GFP can be from any source, with Aequoria victoria and Renilla spp. being particularly useful. In a preferred embodiment the GFP is from A. victoria or from R. reniformis. In other preferred embodiments the GFP is a modified GFP, produced from a modified gene. The modified GFPs can result from mutant selection, for example from chemical mutagenesis, site-directed mutagenesis, substitution of one or more amino acids in a chromophore or elsewhere by genetic manipulation, deletions and/or additions of amino acid residues or domains, fusion to other proteins, or other modifications. The modifications may alter the physical or spectral properties of the GFP to provide for example improved affinity binding; or differential spectral properties, for example, for application in dual FP assays.

The modifications also include those which incorporate properties into the produced GFP molecules which do not deleteriously impact the fluorescent qualities of the GFP and which provide advantageous properties, such as molecular properties useful as ‘handles’ for binding in the trapping step. Examples of such binding properties as may be engineered into a gene for expression as a property of the protein are known in the art. Examples include, but are not limited to addition of: a polyHis tag, a maltose binding domain, a cellulose binding domains, a streptavidin domain, or a strongly immunogenic peptide. One or more of the nucleic acid sequences encoding such properties can be assembled into simple genetic ‘cassettes’ to facilitate incorporation or cloning into GFP-encoding nucleic acid sequences. Affinity binding applications are implemented for the proteins produced from such nucleic acid sequences. In a preferred embodiment, the GFP contains one or more these affinity binding ‘handles’ which are useful for the trapping step.

In one embodiment, the trapping step is a chemical means for binding the FP, to separate it from other components, particularly interfering components in the reaction mixture. Interfering components comprise compounds, materials or substances which autofluoresce, and/or those which scatter and/or quench the fluorescence of the FP in the assay. In one embodiment the trapping step results in a concomitant concentration of the FP by a factor of from about 10⁰-fold to about 10³-fold. In a preferred embodiment, the trapping step results in a concentration factor from about 10³-fold up to about 10⁶-fold or greater. In other contemplated embodiments, a concentration step, such as are known in the art, is used in conjunction with the trapping step.

The trapping step in various embodiments comprises molecular interactions, for example: metal ion affinity binding, ion exchange interactions, or antigen-antibody interactions. In some embodiments, the interacting portions or domains of the GFP involved in the molecular interactions may be exogenous to a native GFP protein, e.g. they may be the result of genetic modification of a gene encoding a GFP molecule to produce a modified GFP. In a preferred embodiment, a chemical means for trapping is bound to, associated with, incorporated in, or part of a matrix or support to allow rapid and specific binding of a GFP or modified GFP.

Reducing the interfering compounds and concentrating the sample allow for a decrease in noise and allow the use of higher intensity excitation wavelength irradiation and generation of higher signals. This results in greatly increased signal-to-noise ratios. In a preferred embodiment, the signal-to-noise ratio is increased about one to many orders of magnitude.

In another embodiment of the present invention, a method for quantifying a fluorescent protein (FP) produced in a cell-based or cell-free expression assay system is provided. The steps of the method are as follows: providing a reaction medium in which to quantify a FP produced during an assay; trapping the produced FP by use of a trapping step for separating the produced FP from one or more interfering components; concentrating the trapped FP into a compact area; irradiating the trapped, concentrated FP with a light source at an excitation wavelength; detecting an emitted light intensity at an emission wavelength; and quantifying the produced FP as a function of the emitted light intensity of the trapped FP.

In a preferred embodiment, the FP is a GFP. The method can be used for quantifying the GFP when it is produced in assays for reporter genes. In one embodiment, screening is based on the promoter-driven expression of GFP. The GFP expressed in these assays exhibits an increased and more uniform level of fluorescence after a step for trapping and concentration, than that exhibited in previously known HTS assays. A preferred GFP is based on that from A. victoria, more preferred is a GFP based on that from a Renilla spp.

The present invention provides methods for the measurement of GFP produced in cell-based or cell-free assay systems. Specifically, the invention provides methods whereby influences on the expression of GFP can be measured with great ease and high sensitivity. The invention also provides these assays as conducted in a high throughput mode, wherein a multitude of samples can be processed and tested.

A typical assay comprises the following steps: cells with an ability to express GFP are incubated in an assay vessel, optionally in the presence of one or more test compounds. The cells are lysed after incubation and expression of GFP, the expressed GFP is released. Released GFP is chemically trapped, resulting in greatly increased GFP concentration and concomitant removal of interfering components. The separated and concentrated GFP is measured based on its ability to emit light of specific wavelength after being excited by ultra high-intensity light of the appropriate wavelength. The data are then analyzed. In a preferred embodiment, the samples are assayed automatically or robotically.

More specifically, one or more suitable assay vessels containing the cells which permanently or transiently possess one or more genes encoding the GFP operably-linked to a transcription promoter are incubated under the appropriate environmental conditions for the time to be tested. Optionally, one or more test compounds, such as a drug candidate to be screened, is included or added to the cells prior to measurement. The cells are then lysed. The cell lysate including the expressed GFP molecules is then subjected to a means, hereinafter referred to as ‘trapping chemistry’ which causes the GFP to become physicochemically trapped or retained. The method removes one or more compounds or substances which negatively or positively interfere with the excitation or emission of fluorescence. These interfering components include many possible assay components, for example: cellular debris, assay debris, contaminants, autofluorescing material, fluorescence scattering material and fluorescence quenching material.

Relative to either the total reaction volume, the cell volume, and/or the cell lysate volume, the GFP is concentrated during this step. This is sometimes hereinafter referred to as ‘microconcentration’. The trapped, microconcentrated GFP is then excited via ultra high-intensity light of the proper wavelength, and the energy is measured at the appropriate wavelength for that GFP. The novel trapping chemistry and microconcentration of the GFP combined with the ultra high intensity light for excitation provided by this invention allows for signal-to-noise ratios of the GFP measurement to be enhanced by several to many orders of magnitude.

Reducing the interfering compounds and concentrating the sample allow for a decrease in noise and allow the use of higher intensity excitation wavelength irradiation and generation of higher signals. This results in greatly increased signal-to-noise ratios. In a one embodiment, the signal-to-noise ratio is increased from about 1-fold to about 10-fold, in a preferred embodiment it is increased from about 10-fold to about 100-fold, in a more preferred embodiment, it is increased from about 100-fold to about 1000-fold, in a still more preferred embodiment, it is increased from about 1000-fold to about 10000-fold, and in a highly preferred embodiment, the signal-to-noise ratio is increased more than about 10000-fold.

In a preferred embodiment, the samples are low volume, less than 1000 μl. Total sample volume may be microconcentrated from about 1-fold to about 100-fold or more, and preferably from about 100-fold to 1000-fold or more, and more preferably from about 1000-fold to about 10000-fold, and still more preferably about 10000-fold or more.

In a particularly preferred embodiment, the GFP is from Renilla reniformis, the reaction volume is less than about 1 ml. the sample is trapped on metal ion affinity chromatography beads, such as Ni-NTA, and concentrated down to less than about 50 μl in a reaction vessel which comprises a microtiter plate well. In one embodiment, the microtiter plate is modified so that the wells are conical with a small flat bottom through which the excitation wavelength can be transmitted and the emission wavelength can be measured. A laser of the argon 488 nm type is used to provide the excitation energy.

In other embodiments the method is used for proteins produced as a result of genetic manipulation—for example from a hybrid protein or protein fusion. Alternatively the GFP may be only slightly modified so as to contain a ‘handle’ such as a binding domain, as for example a polyHis tag.

In one embodiment of the invention, the cells contain a transgene which comprises a promoter of interest, operably-linked to a gene encoding an amino acid sequence of a GFP molecule, such as a Renilla GFP whose encoding DNA has been optimized to express in the species of cells being used. The cells, in multi-chambered vessels, are exposed to or mixed with one or more compounds to be tested, such as drug candidates whose activity is likely to effect the expression of genes driven by the promoter operably linked to the GFP. After suitable incubation conditions (e.g. time and temperature), easily determined by those skilled in the art, the cells are lysed and a suitable trapping chemistry is added to the lysate.

The trapping chemistry allows the GFP to be nearly homogenously removed from its background of cellular debris and sources of autofluorescence, scatter or quenching. The trapped GFP is measured by excitation and emission at wavelengths appropriate to the GFP used. In this homogeneous state, the greater the excitation energy intensity, the greater the emission energy, up to the photostationary state, at which point all GFP molecules in the irradiated field are essentially always excited.

The incubation step comprises environmental conditions such as pH, temperature, O₂ and CO₂ concentrations. Incubation conditions are selected based on the specific cell type being tested and other factors. Such incubation conditions are easily established by one skilled in the art. For example, E. coli cells could be incubated at near neutral pH, at 30° C., under ambient atmospheric conditions.

The lysis step comprises treatment to which the GFP is stable, but to which many other cellular components are labile, such as, but not limited to the range of pH from 5.5-12.6, use of detergents including up to 1% cationic, anionic, zwitterionic or nonionic detergents, use of chaotropic agents such as 8 M urea or 6 M guanadine HCl. Other treatments to which GFP is resistant and whose use is contemplated include proteases, certain water-soluble organic solvents such as ethylene glycol, and temperatures up to 70 C. The stability of GFPs to these and other treatments is known in the art (Ward, 1998 chapter). Other lysis methods known to those skilled in art are contemplated for use in this invention.

The trapping step comprises the addition of a physical or chemical entity which results in the preferential retention or exclusion of GFP relative to other components of the cell lysate such that a relative concentration of the GFP is affected. The trapping chemistry will utilize one or more molecular properties and/or binding properties of the FP; such properties are often used in the purification of proteins and include for example ionic properties, hydrophobicity, 3-dimensional structure, molecular radius, antigenic epitopes for binding with antibodies, electrical properties (e.g. isoelectric point), magnetic properties, and affinity binding properties—such as affinity for particular metal ions or small molecule ligands.

In a preferred embodiment, the trapping chemistry comprises microscopic beads or particles capable of binding the GFP and trapping it on the surface or within the volume of said beads or particles. The beads or particles are selected for their useful properties, such as small size (e.g. 50 μm to 20 μm or less), chemical composition or surface chemistry, including but not limited to glass beads, polystyrene beads, acrylamide beads, agarose beads, ion exchange beads, Nickel-NTA beads (Qiagen Inc, Valencia, Calif.), immobilized metal affinity beads, or beads with immobilized immunospecific moieties or components, for example, anti-GFP antibodies. Beads with immobilized molecules such as biotinlyated compounds, specific carbohydrates or carbohydrate derivatives, specific lipids or lipid derivatives, proteins or protein derivatives, or other molecules for which protein counterparts with specific binding domains exist, are useful for affinity binding proteins which have the functional binding domains present. The methods of introducing functional binding domains, such as those with affinity for these molecules, into proteins, by genetic manipulation of the genes encoding the proteins are well understood. Use of such trapping chemistry in this manner is a novel part of this invention and offers powerful and surprising benefits from the resultant contaminant removal and microconcentration of the GFP.

In another embodiment magnetic particles are used to as part of the trapping chemistry. In this assay, after the lysis, the GFP is pretrapped with magnetic anti-GFP antibodies. The complex of GFP and magnetic anti-GFP can be used to further trap the GFP into a highly concentrated area for measurement via the use of a tiny magnetic source which attracts and collects the GFP-magnetic anti-GFP complex. The collection can be done entirely within the assay vessel, wherein the magnetic source is located in close proximity to the reaction vessel and wherein the magnetic source generates a tightly focused magnetic field of the desired area in which to collect the GFP-magnetic anti-GFP complex, which is then measured by excitation and emission fluorescence. Alternatively it can be envisioned that the magnetic source would remove the GFP-magnetic anti-GFP complex from the assay vessel and redeposit the complex in proximity to a measurement device.

In addition to the foregoing, any other means of trapping or otherwise concentrating the fluorescent molecules is contemplated as being within the scope of the present invention. For instance, as would be understood in microarray technology, surfaces may be prepared for capturing GFP and other fluorescent molecules thereon in microarrays. This is accomplished, for instance, by providing a surface onto which has been deposited, e.g., via inkjet printer, a reactive chemical group or linker that interacts with one or more sites on the GFP or other fluorescent molecule. Such “reactive” surfaces are then contacted with a test sample containing the GFP, which is thereupon captured into an array format. Activated and activatable groups (including photoactivatable groups) for use in protein microarray preparations are well known in the art.

The trapped GFP is measured though the use of extremely high light intensity at the desired wavelength. Since the trapped GFP is free of autofluorescence and other background noise, as well as scatter and quenching, the higher the light intensity, the higher the signal up to the GFP photostationary state. This novel aspect of this invention allows light of many orders of magnitude greater intensity to be used for excitation. The high emission energy which results from this homogeneous GFP, absent autofluorescence and background, gives a signal-to-noise ratio that is again many orders of magnitude greater than existing non microscopic assays.

In one embodiment the invention uses a high intensity light. In a preferred embodiment, an ultra high intensity light source is used to irradiate the trapped GFP. Such light sources are known in the art, for example argon lasers. Light from other sources can be increased in intensity and concentrated with appropriate lens or other optical modification systems. In a preferred embodiment, the light intensity is increased simultaneously in two ways: first the area into which the light signal is focused is made extremely small (‘concentrated’) through the use of optics including but not limited to objective lenses and focusing lenses (‘focused’), and second, through the exclusion of excitation wavelength light from the emission detector by the careful selection of lasers, and the use of optical filters, monochromators and the like. In one application of this embodiment, the trapped GFP is a Renilla GFP, an argon 488 nm laser is used for excitation, focused in a measurement area of less than 20 μm² to 50 μm², and the GFP trapping chemistry is magnetic particles magnetic anti-GFP retained in that size area. Under these conditions, the signal-to-noise ratio is several orders of magnitude higher than in typical cellular gene expression assays incorporating GFP as a measure of expression, resulting in much greater sensitivity.

Additional sources of light which can be considered for generating the high intensity flux required include but are not limited to other lasers, xenon bulbs, mercury vapor lamps, metal halide, halogen, high pressure sodium or other high intensity discharge lights. Choice of the light source will depend upon the intensity and wavelength desired, among other factors.

The present invention also provides a method for high throughput screening assays of compounds affecting the up-regulation of any gene promoter in vivo. In this embodiment, the gene promoter of interest is selected and cloned into a construct containing the coding sequence for a GFP. The construct is used to transform cells of choice by methods which are known to those skilled in the art. The transformed cell lines are then incubated with the compounds to be tested. Lysis and trapping are done as above. The trapped GFP is excited by the high intensity light and the emission is measured. Comparisons are made between the data from control assays and those with added test compounds. Assays which show increased emission at the measured wavelength, relative to control assays, are those which contained compounds which caused up-regulation of the gene promoter being tested.

In another embodiment, in vivo assays for measuring down-regulation of any inducible or constitutive promoter are provided. Various example of such promoters are known in the art; in one embodiment, the transcription promoter comprises one or more transcription promoter properties selected from the group consisting of transgenic, endogenous, constitutive, inducible, single-copy, multiple-copy, developmentally-specific, tissue-specific, cell-type specific, subcellular location-specific, disease-state specific, cell cycle-specific, circadian rhythm-specific, and viral-specific. In this embodiment, the gene promoter of interest is selected and cloned into a construct containing the coding sequence for a GFP. The construct is used to transform cells of choice by methods which are known to those skilled in the art. The transformed cell lines are then incubated with the compounds to be tested.

If the promoter is inducible, the inducer must be added as well as the compound to be tested. Depending on the kinetics of the inducible promoter, and the down-regulation mechanism being considered, the inducer may be added before, during, or after the test compound. Lysis and trapping are done as above. The trapped GFP is excited by the high intensity light and the emission is measured. Comparisons are made between the data from control assays and those with added test compounds.

Addition of inducers and test compounds must each have appropriate controls, as would be understood by one skilled in the art of assay development. Assays which show decreased emission at the measured wavelength, relative to control assays, are those which contained compounds which caused down-regulation of the gene promoter being tested. Such use of down-regulation promoter assays might be considered most appropriate when screening for therapeutic agents for cancers or neoplastic growths or in other situations where a particular gene or gene(s) may be over-expressed or not responding to cellular regulatory signals.

Additionally, compounds which have the ability to up-regulate or down-regulate specific genes under the control of specific promoters may find tremendous use as therapeutic agents, therefore the present invention employs the in vivo expression of Green Fluorescent Protein as a biological target for high throughput screening of such compounds. This novel approach allows screening of putative compounds which affect gene expression. Using the unique properties of the GFP measurement provided, the assays can be performed under conditions where signal-to-noise ratios are exponentially higher than in other assays.

Compounds to be tested include for example drugs, drug candidates, genes, nucleotide or ribonucleotide sequences, gene products, antibodies, immune system or blood components, vaccines, toxins, venoms, enzyme inhibitors, carbohydrates, lipids, proteins, nucleic acids, minerals or their salts, extracts from fungi, microbes, plants, marine life, insects or animals, foods, vitamins, herbal, homeopathic or ayurvedic remedies, traditional medicines from native cultures, or any combinations, parts, fragments, variations or derivatives of the aforementioned compounds.

The invention may be practiced in a cell-free expression system. Normally cell-free expression systems produce such low amounts of product that practitioners have traditionally used radiolabeled amino acids to help quantitate the expressed protein. Due to the extremely high signal-to-noise ratio of the present invention, detection is many orders of magnitude more sensitive than other means of measurement. Therefore this novel aspect of this invention allows its use as a means of monitoring expression in cell-free systems. Examples of widely used cell-free systems include both prokaryotic and eukaryotic systems. E. coli S-30 extract, wheat germ extract and rabbit reticulocyte extract systems are all well known to those skilled in the art. These cell-free systems can be used in batch modes, semi-continuous modes or continuous modes.

The invention may also be used as a rapid and sensitive screening method for mutants. One may look for mutations in specific regulatory sequences which enhance or repress expression of a GFP protein. A GFP encoding sequence can be operably linked to a promoter of interest. The DNA construct can be placed into a vector and used to transform the organism of interest. The expression of the transgene so generated may be controlled by both cis- and trans-acting elements. After mutagenesis by techniques known to those skilled in the art, such cells can be quickly screened by the assays of the present invention. After incubation under suitable conditions, lysis and trapping, the GFP can be measured. Data analysis includes comparison of experimental cells with control cells which contain the transgene but which are not mutagenized. Mutants can be identified which contain mutations which either enhance or repress the expression of the GFP. Identification of mutants which have nonlethal but significant effects on the expression of GFP is likely, but also more subtle mutants with reproducible, but lower magnitude effects on expression may be identified because of the sensitivity of the assay method. These more subtle mutants may be significant in understanding biological control of such regulatory sequences.

From another perspective, such screenings have advantages over many screening methods which require cell division in a selection step prior to identification of mutants. Eliminating the requirement for cell division (for example, growth on plates) might allow one to study genes involved in cell division in a direct, but sensitive manner.

One can also look for mutations in specific sequences using GFP-fusion proteins. In this application, a DNA construct encoding a GFP fused to the protein of interest is constructed. Appropriate methods, well know to those skilled in the art for creating cells expressing GFP-fusion protein are used. The cell lines are then mutagenized by methods known to those skilled in the art and then incubated under conditions allowing expression of the GFP-fusion protein. After lysis and trapping the GFP-fusion product, expression is measured by the excitation and emission of the GFP. Mutants are identified by comparison with the unmutagenized control cells which also express the GFP-fusion protein. Many types of mutants can be rapidly identified with great sensitivity.

Other applications of the invention taught herein include, but are not limited to: application to Fluorescence Activated Cell Sorting (FACS), screening of mutants especially regulatory mutants for promoters of interest operably linked to GFP expression, and application to other fluorescence-based assays known to those skilled in the art.

The invention also provides standard GFPs. In biological testing, standards are required to ensure that instruments are properly calibrated, and also to be sure that assays are linear or predictable in terms of response. Ideally, such standards provide a known amount of response, and should match the analyte in as many respects as possible. The GFP standards provided in the instant invention are extremely useful for calibrating both the instrument and the assay itself. Proper use of such ideal standards is key to being able to make quantitative measurements of differences observed in biological assays.

Examples of standard GFP controls would include trapping GFP from the same organism as selected for the assay, on the same type and size beads, using the same chemistry for trapping, and matching the microconcentration of the standard beads to that of the assay. For ideal standards, matching would be for several other parameters such as is desired, including, but not limited to, the entire excitation spectrum, the entire emission spectrum, the fluorescence quantum efficiency, the molar extinction coefficient, the chemical stability, the photostability and or the fluorescence lifetime. In preferred embodiments, the standard GFP used matches the GFP used in the assay in as many parameters as is practical for the assay being conducted. In a highly preferred embodiment, such standards are created from the exact same batch of GFP used in the assays.

The present invention also provides a novel instrument for measurement of fluorescence in HTS assays. The instrument of this invention contains elements of a steady state fluorescence instrument along with optical elements, or software which could emulate such optic elements such that extremely high light intensity at the desired wavelength(s) can be generated. This instrument while a novel provision of this invention, is in no way intended to limit the other provisions of the invention, as the methods of the invention may be practiced on instruments with only some of the present features. Since the trapped GFP is free of autofluorescence and other background noise, the higher the light intensity, the higher the signal.

In a preferred embodiment, the light intensity produced by the instrument of this invention is dramatically increased in at least one of two or more ways. The light signal is focused in an extremely small area through the use of optics including but not limited to objective lenses and focusing lenses, and computer algorithms which emulate optical components. Another method of increasing the relative light intensity is through the exclusion of excitation wavelength light from the emission detector by the careful selection of lasers, and the use of optical filters, monochromators and the like, or computer software which can control a light source in a manner which emulates optical components. The instrument is also able to utilize one or more light sources of one or more excitation wavelengths such that the excitation wavelength selected is rationally selected for the GFP being used. The instrument consists of a reader, and optionally robotic components for automating the assays such as sample manipulators and sample feeders.

The instrument in one embodiment is capable of testing samples in continuous, semicontinuous and/or batch mode. In another embodiment, the trapped GFP is a Renilla GFP, an argon 488 nm laser is used for excitation, focused in a measurement area of less than 20 μm² to 50 μm², and the GFP trapping chemistry is magnetic particles magnetic anti-GFP retained in that size area. Under these conditions, the signal-to-noise ratio is at least several orders of magnitude higher than in typical cellular gene expression assays incorporating GFP as a measure of expression. The instrument of this embodiment is effective for measuring many GFPs including but not limited to S-65-T, eGFP, YFP, Renilla, Ptilisarcus, and several coral GFPs, and useful, but less effectively so for wild-type GFP and GFPuv.

In order to facilitate the automation of the assay, the methods may be performed in any shape or size vessel whatsoever. In various embodiments, for example, the appropriate vessel comprises any shape, size, or volume and includes tubes, wells, channels, cards, chips, contained drops or droplets, supported drops or droplets, hanging drops or droplets, microtubes, multi-well or micro-well plates, cards, chips or discs, trenches, slots, dots, microarrays, convex or concave ‘bubble’ arrays, microchips, biochips, microfluidic channels, cell sorters, or any microfabricated means of containing, restricting or handling an assay mixture or assay fluid.

The following examples are provided to describe the invention in greater detail. They are not intended to limit the foregoing description of the invention in any way.

### EXAMPLE 1

### The Limits of Detection

Assume spherical bacterial cells (or e.g. trapping particle, bead, and the like), 1 μM in diameter. Such cells expressing GFP are easily detected by fluorescence microscopy.

Given the basic formula for volume (V) of a sphere:
V=4/3πr³, where r=radius of the sphere:

$$\begin{matrix}V = ( 4 / 3 ) ( 3.14 ) {( 0.5 μ M )}^{3} \\ = ( 4 ) ( 0.125 ) {10}^{- 12} {cm}^{3} ( Rounding ) \\ = 0.5 ⨯ {10}^{- 12} {cm}^{3}\end{matrix}$$

If the 1 μM sphere were all (100%) GFP, of density (ρ)=1.3 g/cm³ then the amount (mass (m), in grams, g) of GFP readily visualized by fluorescence microscopy is:
V×ρ=m
(0.5×10⁻¹² cm³)(1.3 g/cm³)=0.65 pg
Converting to moles (MW_{GFP}=27,000)
   (0.65×10⁻¹² g)(1 mole/27,000 g)=2.4×10⁻¹⁷ moles
And using Avagadro's Number to reduce moles to number of molecules:
   (2.4×10⁻¹⁷ moles)(6.022×10²³ molecules/mole)=14.4×10⁶ molecules
   However, recalling that this number is based on the unrealistic assumption that the hypothetical 1 μM sphere (e.g. bacterial cell or trapping particle) consisted 100% of GFP, a more realistic assumption is that only 0.1% of the total E. coli cellular volume is GFP, even in overexpressed systems.

Therefore, in practice, the detection limit is closer to:
14.4×10⁶ molecules/10³=14,400 molecules

It is anticipated that the method is more sensitive than this. Using fluorescence microscopy and the methods of the present invention, it is possible to see green fluorescence from particles as small as 10% the diameter of a bacterial cell.

Assuming therefore, a 0.1 μM sphere, the number of GFP molecules is reduced in number by the cube of 0.1, i.e. 1000 fold.
14,400 molecules/1000=14.4 molecules

In conventional fluorescence assays for GFP, the best sensitivity we have obtained using any of three standard fluorometers is 5 pmoles per assay. Calculating molecules:
(5×10⁻⁹ moles)(6.022×10²³ molecules/mole)=3×10¹⁵ molecules

Using the methods of the present invention, assuming 1 μM diameter bacterial cells that are 0.1% GFP by volume (or the equivalent amount of GFP trapped in an area of that size, for example on beads) would improve assay sensitivity by the following factor:
3×10¹⁵ molecules/conventional assay÷1.44×10⁴ molecules/GFP trapping button=3×10¹⁵/1.44×10⁴ ≈2×10¹¹ times more sensitive.

In other words, on a mass basis, the detection limit of the methods of the present invention, is 0.2 trillion times more sensitive than conventional assays.

If the sensitivity limit of a standard fluorometric plate reader is 5 pmoles of GFP, we can lower that limit by a factor of 0.2 trillion fold by trapping the GFP into a 1 μM spherical area.
5×10⁻⁹ moles/0.2×10¹²=10×10⁻²¹ moles=1×10⁻²⁰ moles detectable
Converting again to molecules:
   (1×10⁻²⁰ moles)(6.022×10²³ molecules/mole)
=6×10³ molecules per microtiter well (200 μl volume) trapped into a 1 μM volume.
=6000 GFP molecules per assay.
=6 cells each containing 1000 GFP molecules
   or 60 cells each containing 100 GFP molecules
   or 600 cells each containing 10 GFP molecules
   or 6000 cells each containing 1 GFP molecule

### EXAMPLE 2

### Detection Limit on Conventional Fluorometers

Calibration curves were performed on three commercial fluorometers optimized for GFP detection, using GFP expressed in E. coli cells. The fluorometers include a Turner 110 filter fluorometer, a Hoefer TKO 100 fluorometer, and a computer-operated Thermo Lab Systems MFX fluorometric microplate reader.

The detection limit for the wild-type GFP on the Turner 110 was 5 pmoles per assay. To minimize scatter, the fluorometer was set to the 10× slit setting and E. coli cells were at OD₆₆₀ of about 0.25—i.e. the determined limit is for nonturbid samples only. The sensitivity for the Hoefer TKO 100 was determined to be 12 pmoles per assay. This detection limit was essentially unaffected by scatter caused by the E. coli cells. The Thermo Labs MFX was determined to be capable of detecting GFP down to 10 pmoles per assay, a result also virtually not influenced by scatter.

By comparison, when the GFP from cells in a 200 μl microplate well are released (e.g. with a suitable lysis cocktail) and trapped efficiently onto 1 μm sized area, or more likely a volume of 1 μm (a micro “button” of this size can be accomplished by the trapping methods of the present invention), then by combining the optics of a fluorescence microscope with the convenience and speed of a microplate reader so as to selectively view that “button” with intense light at the desired wavelength, an increase of sensitivity of 2×10¹¹ can be achieved. Even if the trapping volume were required to be as large as 5 μm³, the methods of the instant invention provide an increased sensitivity of 1.5×10⁹ fold over the microplate reader.

A single 5 μm C₄-derivitized silica bead saturated with GFP is readily visualized by the unaided eye; i.e. surprisingly, the human eye is more sensitive to microconcentrated GFP than the a computerized microplate scanner is to the same amount of GFP distributed in a volume of 200 μl in a microtiter well. It is therefore expected that such surprising results can readily be achieved by employing the methods of the instant invention coupled with the instrument as disclosed herein, and that these results and methods have great utility for high throughput screening.

### EXAMPLE 3

### Trapping GFP by Hydrophobic Interaction

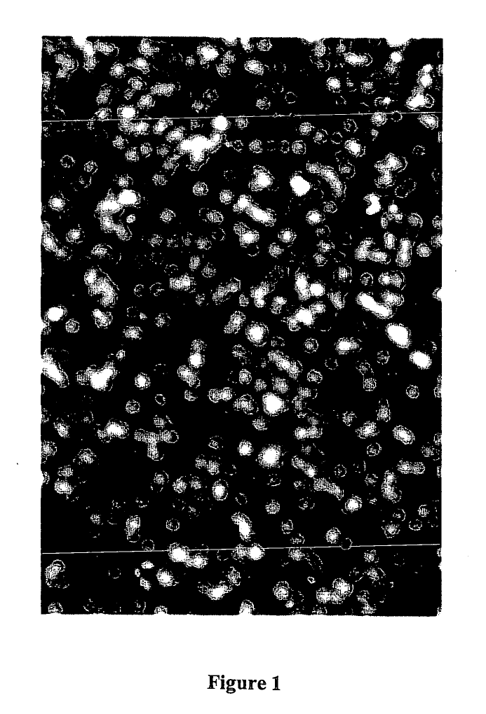

C₄ derivatized silica beads, 5 uM in diameter, (reversed phase HPLC beads from BioRad product number 125-0134) were used to trap GFP by hydrophobic interaction. The C₄ (n-butyl)-derivitized silica based beads were dispersed in methanol and then added to an aqueous solution of wild-type recombinant GFP. The GFP bound immediately to the beads by hydrophobic interaction, producing fluorescently labeled beads so intense in their fluorescence that, despite their tiny size, they can be easily viewed, individually, by the unaided eye on the surface of a hand held long wavelength (365 nm) UV lamp. In this case, the beads were viewed with a BH-2 Olympus fluorescence microscope using a high pressure mercury arc lamp and a blue excitation filter selected for optimal excitation of fluorescein. Results are shown in FIG. 1.

In all micrographs presented in this patent application, the ocular lens was 10×. In this particular view (see FIG. 1), the objective lens was 20×, producing a total field 0.78 mm in diameter (the camera restricts the field by about 50%). Photography was performed with the Olympus PM-10ADS Automatic Photomicrographic System using an Olympus C-35-AD-2 camera. White spots in the field are beads that bound so much GFP as to overexpose the film. Exposure time was 0.75 sec.

### EXAMPLE 4

### Trapping GFP by Ionic Interaction

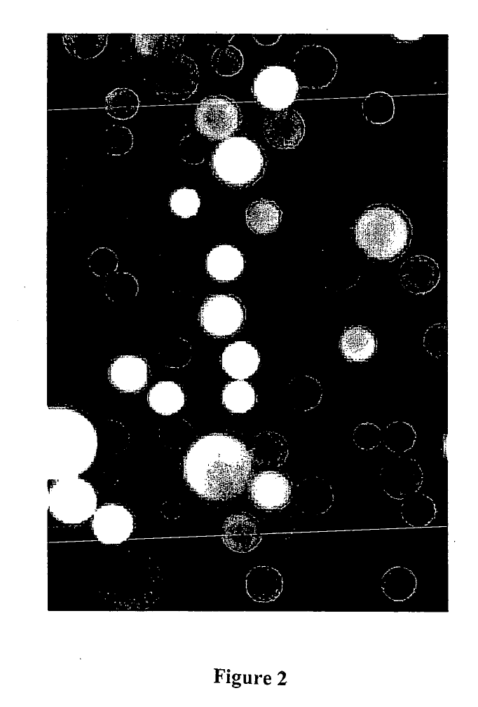

FIG. 2 depicts DEAE (diethylaminoethyl) Sepharose Fast Flow chromatography beads from Amersham BioScience having an average particle size of 90 uM (Amersham product number 17-0709-01). In this view, the DEAE beads were exposed to wild-type recombinant GFP at low ionic strength so as to promote ion exchange interaction of GFP with the DEAE functional group. Differential exposure of the beads to GFP was created by treating some beads with high concentrations of GFP and others with very low concentrations of GFP. The differentially exposed beads were then mixed and viewed at low power (10× objective) generating a total field diameter of 1.6 mm. Heterogeneity of bead size and variable degree of GFP binding is evident. The white beads (highly overexposed) are those with high levels of bound GFP while the green and teal colored beads represent those with lower levels of bound GFP. Exposure time was 0.45 sec.

### EXAMPLE 5

### Microdetection of Fluorescent Protein in Individual E. coli Cells

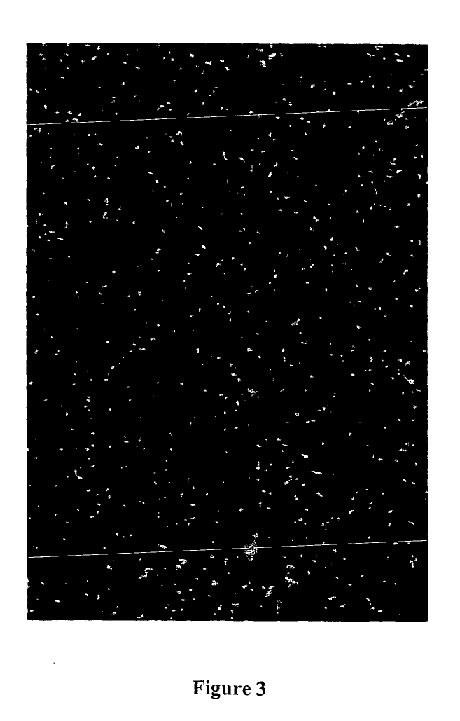

FIG. 3 depicts a field of whole, living E. coli BL-21 cells expressing the red coral fluorescent protein, DsRed 1 (Clontech), under the control of the lac operon using kanamycin antibiotic for selective pressure. Bacteria were removed from a petri dish with a sterile toothpick and spread onto a microscope slide in as thin a layer as possible. Although the plate from which these cells were taken had been stored in the refrigerator for 30 days, the addition of water to bacterial smears from this plate resulted in immediate, vigorous flagellar movement that made still photography impossible. To avoid such movement, no water was added. Excitation was with a green filter selected for optimal excitation of rhodamine B. Individual bacteria in this field are assumed to have average diameters of about 1 uM. Those bacteria that appear white are centered with respect to the focal plane of the exciting light, and thus are so fluorescent as to overexpose the film. Those that appear red are situated slightly above or below the focal plane. This photomicrograph was taken through the 40× objective lens with an exposure time of 1.23 sec.

### EXAMPLE 6

### E. coli-Expressed Cloned Red Coral Fluorescent Protein Traped by Hydrophobic Interaction

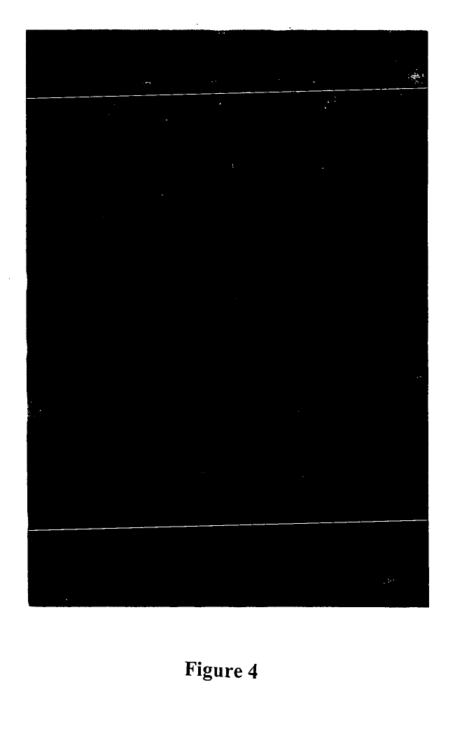

FIG. 4 depicts a field of C₄-derivitized 5 uM silica beads, as in the case described above in Example 3 (and seen in photomicrograph of FIG. 1). In this case, the beads were used to trap pure DsRed 1 fluorescent protein. DsRed 1 fluorescent protein originates from coral but here was expressed in E. coli BL-21 cells as described in the Example 5.

DsRed 1 is strongly attracted to such hydrophobic surfaces and remains bound in a stable state for months to years. Excitation was with the rhodamine B-optimized filter and viewing was with the 20× objective lens. Exposure time was 0.62 sec.

### EXAMPLE 7

### Trapping Can Distinguish Differentially Fluorescent Proteins With Great Sensitivity

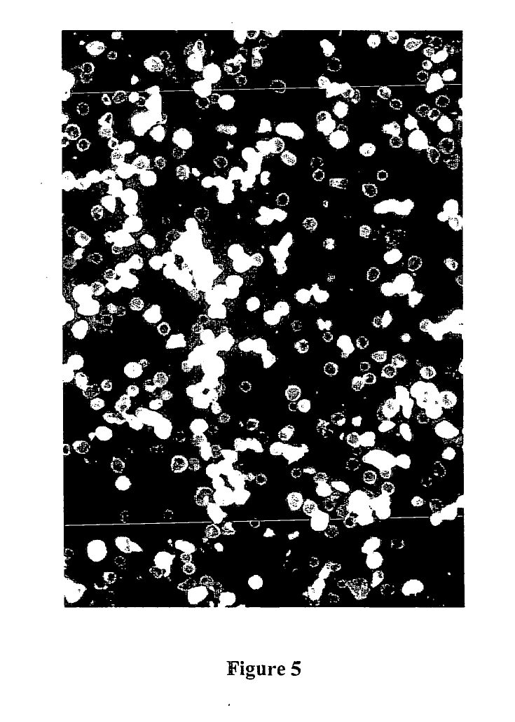

FIG. 5 depicts C₄-derivatized 5 uM silica beads containing varied amounts of either the wild-type recombinant green fluorescent protein or the coral-derived DsRed 1 protein expressed in E. coli. The beads were differentially exposed to GFP or to DsRed 1 and then mixed together. With the blue exciting light and relatively long exposure time, the DsRed 1 protein appears yellow fluorescent while most of the GFP-containing beads correctly fluoresce green. A 20× objective lens was used for viewing. Exposure time was 0.76 sec.

FIG. 8 also depicts differentially labeled beads (i.e. beads with different fluorescent proteins trapped on them) similar to those shown in the photomicrograph of FIG. 7, except the magnification is increased. The view is that with the 40× objective lens. Exposure time was 1.23 sec.

It is expected that the performance as demonstrated by the foregoing examples that the methods of the present invention offer an advantage in that they are relatively easy, offer high signal-to-noise fluorescence, allow images to readily be captured as a way of storing, comparing, or analyzing results (such images can also be digitized for additional analysis), allow use of fluorescent protein trapping chemistries, beads and cells on a size scale ranging, in various embodiments from preferably 100 μm diameter, more preferably 20 μm diameter, even more preferably 10 μm diameter, and most preferably, 1 μm diameter or less.

No special filters are required for the methods or to obtain images of such analyses. For the above examples, two general purpose optical filters were used. Specialized interference filters that have been designed specifically for the fluorescence microscopic examination of all sorts of GFP variants are known to those of skill in the art. It is anticipated that such filters would further improve data collection and noise discrimination.

The data presented in the examples set forth above exemplify that by trapping fluorescent proteins onto “buttons” as small as 1 μm diameter, one can easily and sensitively distinguish signal from noise using the standard optics of a simple fluorescence microscope. In utilizing the fluorescent protein trapping technique in conjunction with the convenience of a modified fluorimetric plate reader and the optics of a fluorescence microscope HTS analysis of GFP-producing cells can be improved by as much as 0.2 trillion-fold, or more depending on the specifics of the trapping method and optics selected.

The present invention is not limited to the embodiments described and exemplified above, but is capable of variation and modification within the scope of the appended claims.

1-36. (canceled)

37. A apparatus for measuring fluorescence of a sample comprising at least one fluorescent protein (FP); the apparatus comprising:
at least one light source for providing high intensity light at an excitation wavelength of the FP, an optical means for focusing light emitted by the light source onto a sample comprising the FP, and a detecting means for detecting fluorescence of the FP by measuring emitted light intensity at an emission wavelength, wherein the apparatus is adapted for receiving a vessel comprising at least one sample wherein, for each such reaction, the FP is trapped using a trapping chemistry, the trapped FP is irradiated with the light source, and the detector measures light emitted by the trapped FP.

38. The apparatus of claim 37 wherein the optical means focuses the light into an area less than about 50 μm².

39. The apparatus of claim 37 wherein the optical means focuses the light into an area less than about 20 μm².

40. The apparatus of claim 37 wherein the light source is an argon laser providing high intensity light at an excitation wavelength at about 488 nm.

41. The apparatus of claim 40 wherein the FP is S-65-T, eGFP, YFP, Renilla, Ptilisarcus, coral GFP, wild-type GFP, or GFPuv.

42. The apparatus of claim 41 wherein the trapping chemistry comprises magnetic particles comprising antibodies to the FP.

43. The apparatus of claim 37 further comprising a means for handling samples continuously.

44. The apparatus of claim 37 further comprising robotic components for automation.

45. The apparatus of claim 37 wherein the optical means comprises one or more of objective lenses, focusing lenses, and optical component emulation algorithms.

46. The apparatus of claim 37 wherein the vessel comprises at least one tube, well, plate, channel, chip, card, disc, channel, trench, slot, dot, array, microfluidic chamber, contained drop or droplet, supported drop or droplet, or hanging drop or droplet.

47. The apparatus of claim 37 which detects less than about 5 picomoles of FP per assay.

48. The apparatus of claim 37 which detects less than about 10×10⁻²⁰ moles of FP.
